(19) United States
(12) Patent Application Publication
(10) Pub. No.: US 2017/0050369 A1
(43) Pub. Date: Feb. 23, 2017

(54) HELICALLY WOUND FLUID-FLOW HOSE

(71) Applicant: inSleep Technologies, LLC, Weston, FL (US)

(72) Inventor: Martin W. Kerber, Eden Praire, MN (US)

(21) Appl. No.: 14/832,699

(22) Filed: Aug. 21, 2015

(51) Int. Cl. B29C 55/00; F16L 11/16; A61M 16/08; F16L 11/08; F16L 57/02

(52) U.S. Cl. CPC B29C 55/00; F16L 11/081; F16L 57/02; A61M 16/0875; F16L 11/16; A61M 2202/0241; B29L 2023/005

(57) ABSTRACT

A fluid supply hose having one or more support ribs, such as a helical support rib that forms a coil and/or individual support rings, of a relatively stiff thermoplastic material, and a thin web or wall of thermoplastic material that extends between the inside, outside or both of the ribs. The hose is exposed to an annealing process comprising: bringing the hose to a fully compressed position where the ribs are brought close together; heating the hose; while the hose is hot, extending the hose axially to a partially extended position where the distance between adjacent winds of the rib or rings of the hose when the hose is in the partially extended position is between about 20%-95% of the distance between adjacent winds of rib 20 or rings 40 of the hose when the hose is in the fully extended position; and allowing the hose to cool in the partially extended position.

### TECHNICAL FIELD

This invention relates to the field of fluid carrying tubes and hoses, and more particularly relates to tubes and hoses that are reinforced to resist compression, yet still remain flexible and relatively soft.

### BACKGROUND OF THE DISCLOSURE

The present invention relates to flexible and easy-to-stretch hoses that are crush resistant and well suited to provide a flow of fluid, such as a supply of air, anesthesia gas or gas-carried medication to a patient's face mask, nasal mask or tracheotomy tube for a variety of purposes such as anesthesia, life support or medication delivery, or to help prevent sleep disordered breathing.

It is well known to use fluid-carrying hoses to convey fluids (e.g., air) from an airflow source to an airflow destination. In many such applications, it is desirable that the hose be both soft, flexible, stretchable and compressible axially, yet strong enough to resist substantial crushing and/or kinking forces.

Some prior crush resistant plastic hose proposals intended for medical use are produced by extruding a thin web of plastic material to provide a connecting wall extending between adjacent coils of a helical rib of plastic. This connecting web may take a wavy form, or may incorporate accordion-like folds, that enable the hose to extend and contract in an accordion-like manner to give the resulting hose a measure of flexibility.

Although the hoses just described may be effective in delivering fluids to a destination, the nature of the extrusion process used to produce these hose products typically causes the resulting hoses to exhibit a high degree of torsional stiffness and an otherwise diminished degree of flexibility due to the orientation of the molecules that form not only the thin wavy wall but also the helix that enhances the crush resistance of the hose. For example, undue torsional stiffness can cause a patient's face or nasal mask to lift off the face during movements of the patient's head, thereby adversely affecting the pressure within the breathing circuit during therapy. The stiff nature of existing products also may cause undesirable stress on a tracheotomy tube during patient movement, and can render difficult, and therefore uncomfortable, head movements of a patient, leading to reduced patient compliance. Also, undue hose stiffness may cause the headgear to which the hose is attached to dislodge from its position on the patient's head.

Ideally, a substantially leak proof seal should be maintained between the patient interface and the face of the patient. Forces applied to the patient interface, such as tube drag or the weight of the mask, or components attached to the mask, tend to disrupt the seal formed between the patient interface and the patient.

Various solutions have been proposed for reducing the undesirable forces that may be applied to a mask or headgear, including tube drag. Some of these solutions include a rotating or swiveling connector to connect the air delivery hose and the patient headgear or interface. The rotating or swiveling connector allows some form of rotation before the tube pulls on the patient headgear or interface, potentially disrupting the seal. Some prior art swivel arrangements use tight tolerances, which might result in friction in the movement of the swivel elbow, thus reducing the mobility and flexibility of the swivel joint.

Another solution which has been proposed to reduce the application of undesirable forces on the patient interface where a trunk-type tube is used to supply air to the interface is a headgear to provide stability to the patient interface and maintain the seal during the application of the forces, including tube drag. The headgear assembly may be designed such that stabilizing straps are provided at an angle with respect to the patient interface and the face of the patient to counteract the undesirable forces. In one known mask assembly, the headgear includes a cap portion with four straps. In use, the cap portion engages the back of the patient's head and two lower straps extend between the cap portion and a nasal mask while the two upper straps extend between the cap portion and a forehead support. Such headgear assemblies, however, are typically too weak to be able to resist the substantial dislodging forces imposed on the interface by the trunk hose.

Another solution for offsetting tube drag and other undesirable forces on the patient interface includes clips that connect the air delivery conduit or hose to the patient's clothing, such as the patient's night clothes. Clips have also been used to connect the air delivery conduit or hose to a stationary object, such as the patient's bed, to remove or reduce tube drag affecting the mask seal. Neither of these arrangements is deemed acceptable.

It has also been proposed to provide a short, more flexible, “transition” tube between the air delivery conduit and the ultimate delivery tube, to provide extra axial and torsional flexibility to the air delivery conduit to reduce pull on the mask.

Tubes or hoses that are reinforced to resist crushing and kinking forces are known in the art. Many efforts have been made to provide resistance to such forces. In some approaches, such as disclosed in U.S. Pat. No. 3,927,695, the tube is wrapped in multiple layers of silicone.

In other approaches, such as disclosed in U.S. Pat. Nos. 6,394,145 and 8,453,681 and U.S. Patent Application Nos. 2014/0207115 and 2009/0078259, a helical structure or independent rings are used to reinforce the tube.

Thermoplastic is a common material out of which to manufacture such hoses, due to its low cost and compatibility with a wide variety of application needs. Many reinforced tubes are formed by extrusion and/or heating the thermoplastic web materials on a mandrel. In some approaches, such as disclosed in U.S. Pat. Nos. 3,927,695 and 8,453,681 and U.S. Patent Application No. 2014/0207115, the web and helical rib or independent rings are placed on a mandrel and heated to form the structure of the hose. The web extends as a straight, fairly rigid, structure between the coils of the support rib or rings.

After the hose is formed, in some approaches, such as disclosed in U.S. Pat. No. 8,453,681 and U.S. Patent Application No. 2014/0207115, the helical structure or independent support rings are compressed and an annealing process of heating and cooling is applied while the hose is in the fully-compressed position. As the annealing process is completed (while the hose is in the fully-compressed position), the molecules of the material of the hose relax and take on a new orientation, with the at-rest orientation of the hose being the fully compressed-position. By applying a force, the hose can then be stretched out to the fully-stretched position. However, the hose will constantly apply a resistive force to return to the fully-compressed position.

Tubes and hoses are used in various applications throughout the medical field. In many of the medical applications, the hoses must remain connected to patients despite the movements of the patient. Thus, such hoses must be able to stretch and bend, so that the patient remains connected to the hose. At the same time, the hoses must be strengthened to avoid crushing and kinking that would otherwise block fluid flow.

For example, in the treatment of sleep-related disorders, such as snoring and sleep apnea, the hose must remain connected to the CPAP system while the patient sleeps. The CPAP system must be able to provide a constant airflow to the patient, so that the patient's airways remain open, regardless of the movements of the wearer during sleep. Thus, the hose connecting the CPAP system to the patient must be flexible. Additionally, the hose must be able to resist radial compressive forces that would otherwise restrict the airflow supplied to the patient.

Currently, standard hoses connecting a CPAP system to a patient subjects the patient, and those around him or her, to much discomfort. The hose overly restricts the movement of the patient because the hose resists movement of the patient. Thus, the patient and the patient interface are subject to resistive forces associated with the hose any time the patient moves while sleeping.

Therefore, it is an object of the invention to provide a hose that exhibits comfort and flexibility, so that the movements of the wearer are not overly restricted or resisted.

It is another object of the invention to provide a hose that is reinforced to resist radial compressive forces that would otherwise restrict fluid flow. By “radial compressive forces” is meant forces leading to crushing and/or kinking of the hose.

It is also an object of the invention to provide a fluid supply hose that is not at or near its fully compressed orientation when at equilibrium, so that the hose can be both axially stretched and compressed, as well as bent and twisted, by movements of the user without undue forces being exhibited on the user, while the hose simultaneously resists crushing and kinking forces.

These and other objects are achieved by the configuration and arrangement of component parts of tubes as shown and described herein.

### SUMMARY OF THE INVENTION

In one form, the invention comprises a fluid supply hose having one or more circumferential support ribs, such as a helical support rib or, alternatively, individual support rings that form a coil, of a relatively stiff thermoplastic material, and a thin web or wall of thermoplastic material that extends between the inside, outside or both of the helix/coils. The hose is exposed to an annealing process comprising: bringing the un-annealed hose to its fully compressed position such that the rings or winds of the helix are brought close together; heating the hose; while the hose is hot, extending the hose axially to a partially extended position; and allowing the hose to cool in the partially extended position. The process results in a hose that is softer and more flexible than the hose was originally, which can be stretched, compressed, bent and/or twisted to absorb external forces while substantially resisting crushing and kinking forces.

Other embodiments of the invention are disclosed below and/or disclosed in the appended claims.

### BRIEF DESCRIPTION OF THE DRAWINGS

FIG. 1 is a perspective view of a first type of an exemplary hose constructed in accordance with the principles of this invention, in its relaxed state prior to heat treatment.

FIG. 2 is a front elevational view of the hose in a compressed state prior to and during heat treatment.

FIG. 3 is a cross-sectional front elevational view of the hose in the compressed state.

FIG. 4A is a front elevational view of the hose in a partially stretched state during heat treatment.

FIG. 4B is a close up of the area of detail shown in FIG. 4A.

FIG. 5A is a cross-sectional front elevational view of the hose in the partially stretched state.

FIG. 5B is close up of the area of detail shown in FIG. 5A.

FIG. 6 is a perspective view of the hose in its relaxed state after heat treatment.

FIG. 7 is a flow diagram of an exemplary inventive process of the invention.

FIG. 8 is a perspective view of second type of an exemplary hose constructed in accordance with the principles of this invention, in its relaxed state prior to heat treatment.

FIG. 9 is a front elevational view of the second type of hose in a compressed state prior to and during heat treatment.

FIG. 10 is a cross-sectional front elevational view of the second type of hose in the compressed state.

FIG. 11A is a front elevational view of the second type of hose in a partially stretched state during heat treatment.

FIG. 11B is close up of the area of detail shown in FIG. 11A.

FIG. 12A is a cross-sectional front elevational view of the second type of hose in the partially stretched state.

FIG. 12B is close up of the area of detail shown in FIG. 12A.

FIG. 13 is a perspective view of the second type of hose in its relaxed state after heat treatment.

### DETAILED DESCRIPTION OF PREFERRED AND OTHER EMBODIMENTS

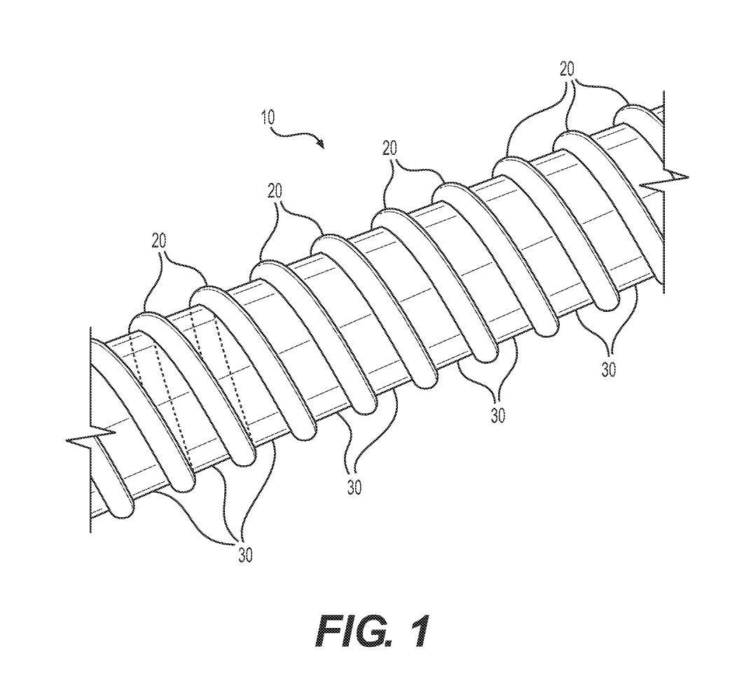

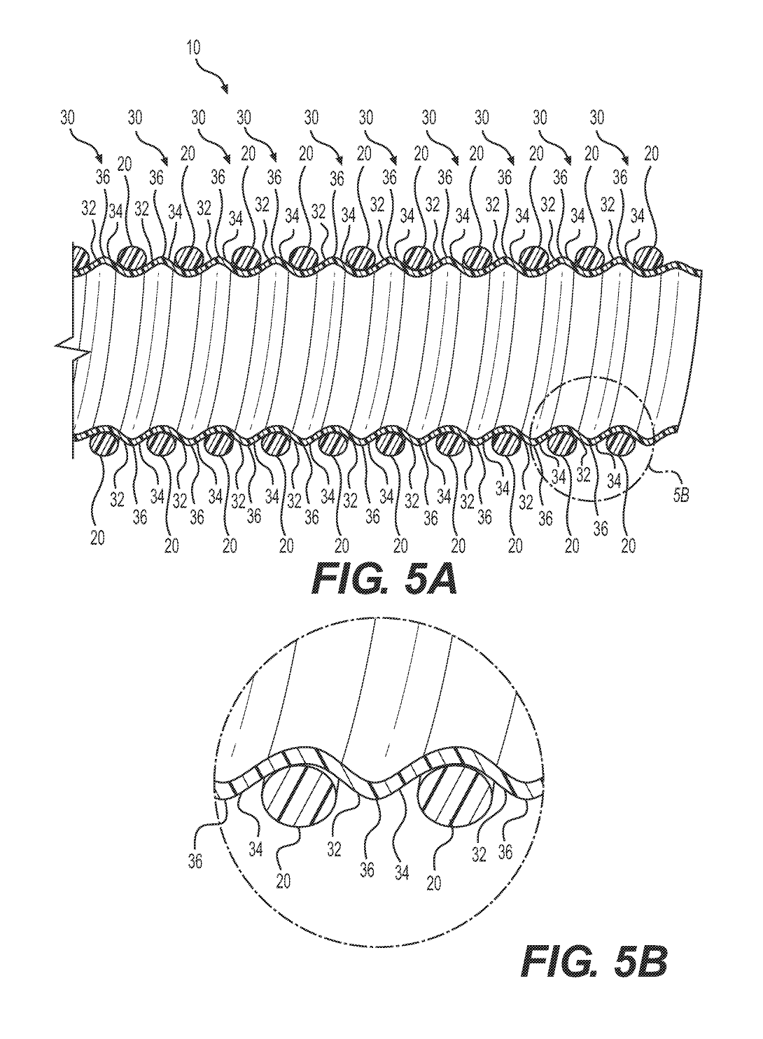

Referring first to FIGS. 1-7, a length of flexible, stretchable, crush resistant hose in accordance with one form of the present invention is indicated generally by the numeral 10. This particular embodiment of hose 10 includes one or more helical support ribs 20 that form a rib of a relatively stiff thermoplastic material, and a thin web or wall 30 of plastic material that extends between the inside diameter, outside diameter, or both, and/or between the winds, of the one or more helical support ribs 20.

Although the hose 10 can be formed in a variety of ways which are considered to be encompassed within the invention, one manufacturing technique which can be employed calls for the material(s) that forms the rib 20 and the thin web or wall 30 to be extruded, either concurrently as separate extrusions that are promptly combined (e.g., bonded or welded together) while still hot following extrusion, or as a single extrusion that forms the helical rib 20 together with an integral sheath or thin web or wall 30.

Alternatively, the hose 10 can be formed by applying the one or more helical support ribs 20 to a mandrel M, and prior thereto, or subsequent thereto, applying the thin thermoplastic web or wall 30 to the mandrel M so that the rib(s) 20 and wall 30 abut one another.

Hose 10 may be formed of any suitable material, including but not limited to PVC, TPU, PPE, TPE, ABS and other thermoplastic materials and reasonable equivalents thereof, to form what results in or amounts to an integral assembly that typically exhibits no remaining borders between adjacent portions of the bonded or welded materials. The terms “welded” and “bonded,” and the terms “welding” and “bonding,” are used interchangeably, with no intended differences of meaning intended therebetween.

The hose, once formed, will have an equilibrium, relaxed or at rest position such as that shown in FIG. 1, in the case shown being in a fully extended position, in the absence of external forces being applied to the hose. The hose 10 may be rendered in a position other than the fully extended position prior to annealing. The hose can be heat treated to alter its properties, such that the relaxed position can be changed and the hose generally rendered softer and more supple.

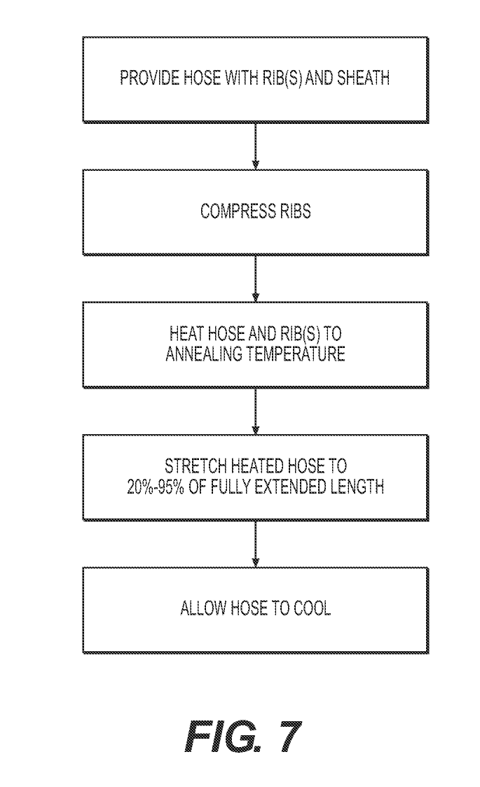

The hose 10 is rendered flexible, stretchable and axially compressible by an annealing process. A representative annealing process for treating hoses in accordance with this invention is depicted in the flow diagram shown in FIG. 7, which comprises the steps of:
providing a hose having one or more helical support ribs interconnected by a thermoplastic web;
bringing the hose to a fully compressed position such that winds of the one or more helical ribs are brought close together;
heating the hose;
while the hose is hot, extending the hose axially to a partially extended position; and
allowing the hose to cool in the partially extended position.

In an embodiment, the distance between adjacent of the helical rib(s) 20 when the hose is in the partially extended position is between about 20%-95% of the distance between adjacent winds of the helical rib(s) 20 when the hose is in the fully extended position.

In another embodiment, the distance between adjacent winds of the helical rib(s) 20 when the hose is in the partially extended position is between about 40%-95% of the distance between adjacent winds of the helical rib(s) 20 when the hose is in the fully extended position.

In yet another embodiment, the distance between adjacent winds of the helical rib(s) 20 when the hose is in the partially extended position is between about 60%-95% of the distance between adjacent winds of the helical rib(s) 20 when the hose is in the fully extended position.

In a further embodiment, the distance between adjacent winds of the helical rib(s) 20 when the hose is in the partially extended position is between about 60%-80% of the distance between adjacent winds of the helical rib(s) 20 when the hose is in the fully extended position.

In a still further embodiment, the distance between adjacent winds of the helical rib(s) 20 when the hose is in the partially extended position is about 80% of the distance between adjacent winds of the helical rib(s) 20 when the hose is in the fully extended position.

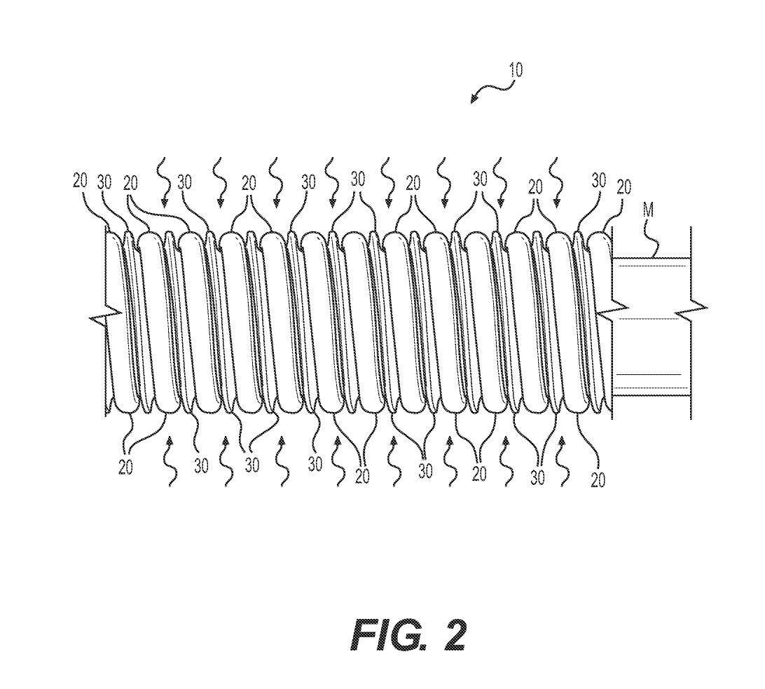

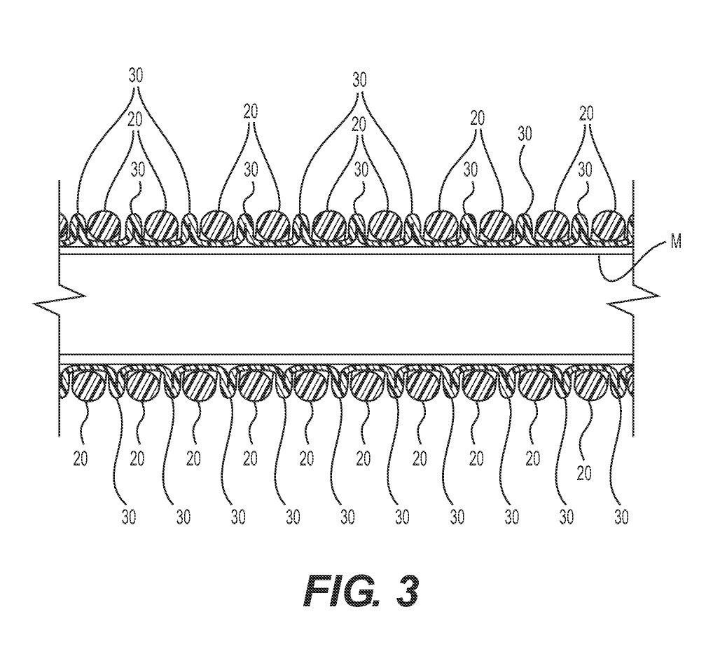

FIG. 1 shows the fully-stretched hose 10 prior to annealing. FIG. 2-3 show the hose 10 in the fully compressed position during which it is heated to an annealing temperature.

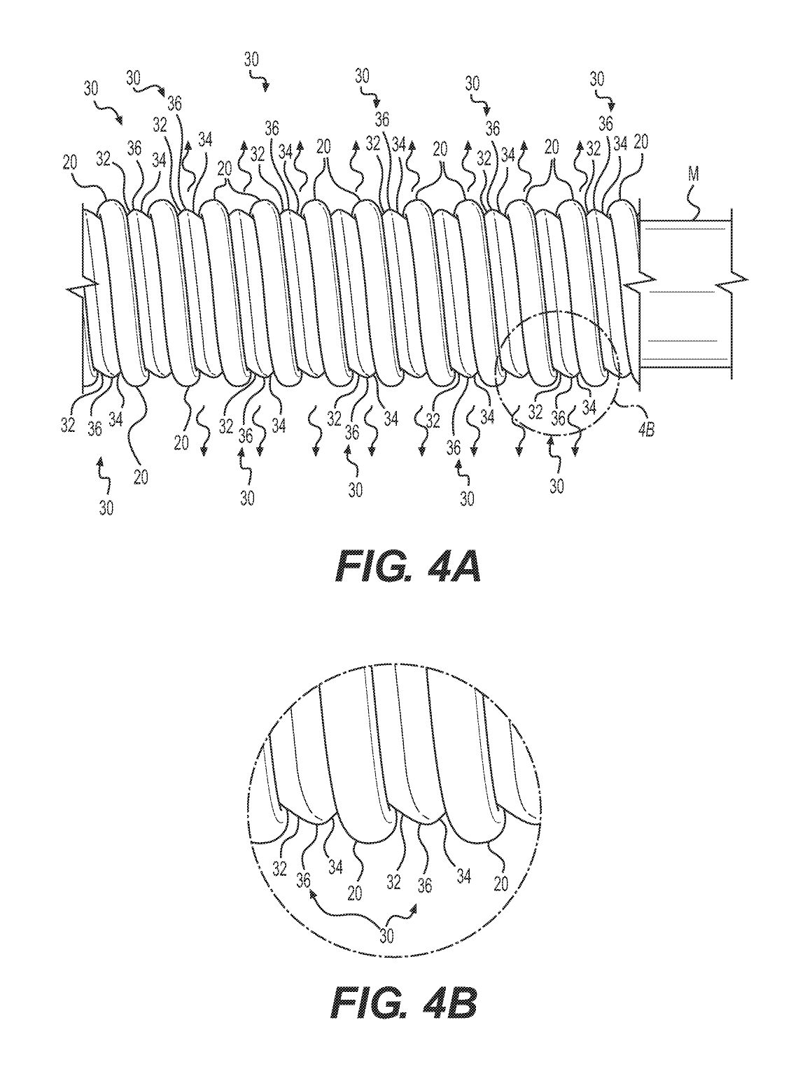

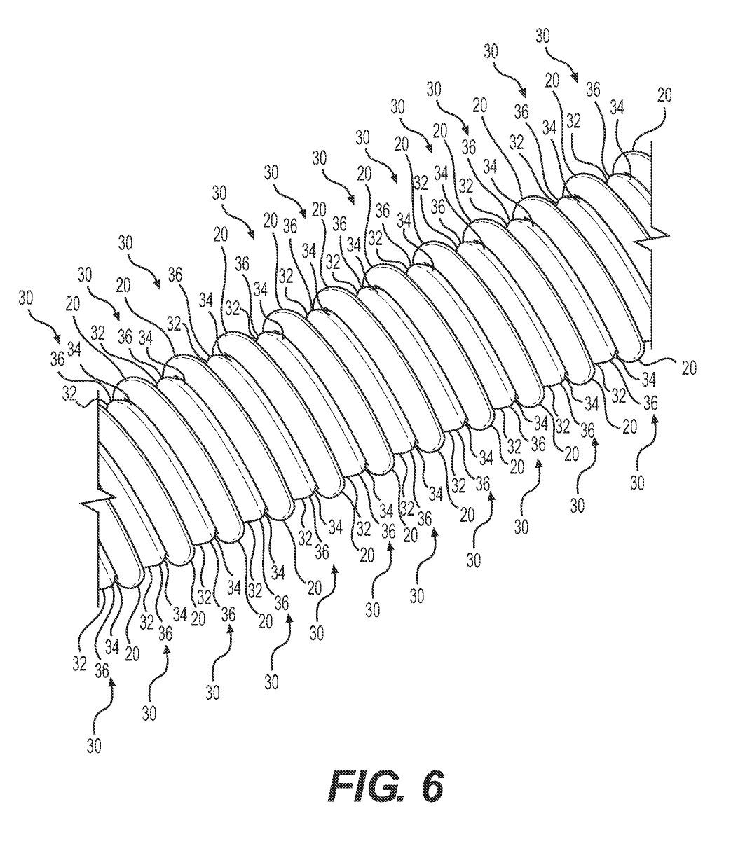

FIGS. 4A and 6 show the hose in the partially-extended position during and after cooling.

FIG. 6 shows the hose 10 in the partially extended or stretched position, which is now its at-rest or equilibrium orientation in the absence of external forces, after being cooled.

FIG. 7 is a flow chart of an exemplary process for treating hoses of the type employed in connection with this invention.

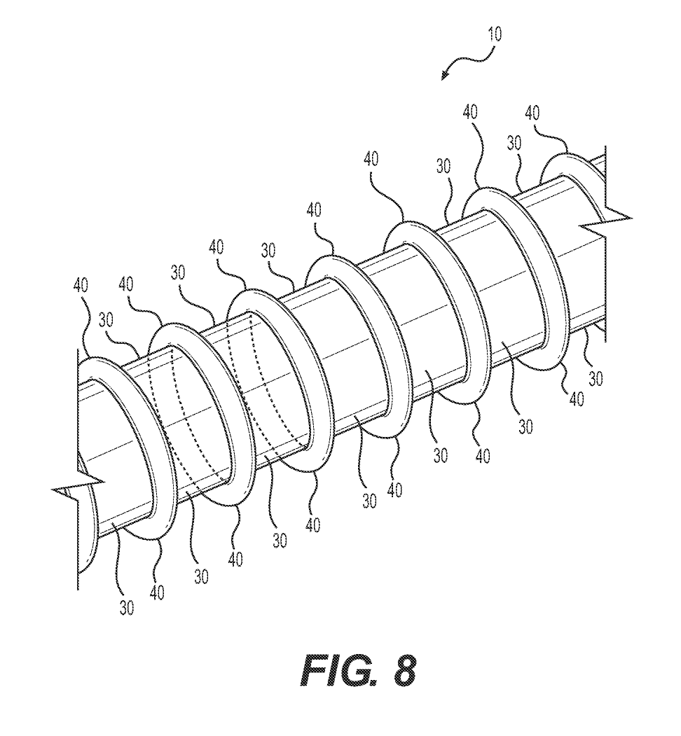

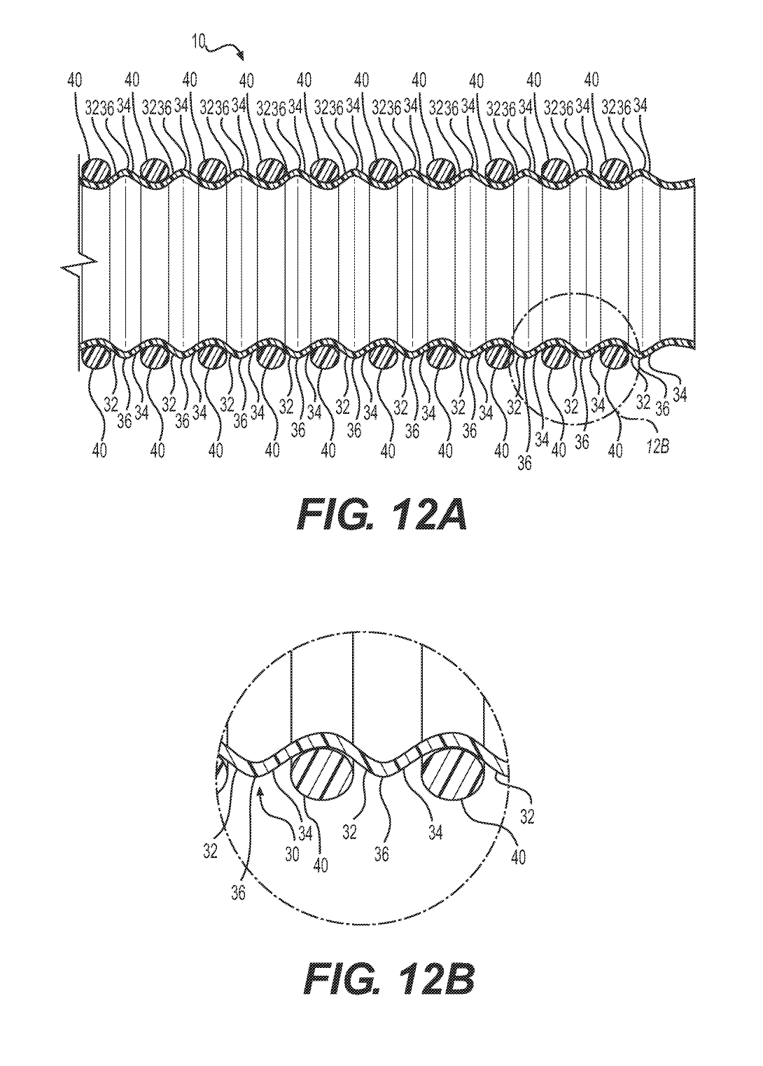

Referring to FIGS. 8-13, a length of flexible, stretchable, crush resistant hose embodying features of the present invention is indicated generally by the numeral 10. The hose 10 includes one or more individual support rings 40 that form a skeleton of a relatively stiff plastic material, and has a thin web or wall 30 of plastic material that extends between the inside, outside or both of rings 40, or between the individual rings 40. Although the hose 10 of this embodiment can be formed in a variety of ways, a preferred manufacturing technique which can be employed calls for the materials that form the rings 40 and the thin web or wall 30 to be extruded, either concurrently as separate extrusions that are promptly combined (e.g., bonded or welded together) while still hot following extrusion, or as a single extrusion that forms the rings 40 together with an integral thin web or wall 30.

Alternatively, the hose 10 can be formed by applying the one or more individual support rings 40 to a mandrel M, and prior thereto, or subsequent thereto, applying the thin thermoplastic web or wall 30 to the mandrel M so that the ring(s) 40 and wall 30 abut one another.

In an embodiment, the invention is directed to a flexible, stretchable, axially compressible fluid-carrying hose 10, the hose 10 including one or more relatively rigid thermoplastic support rings 40 making up a portion of the hose, the hose also including a thermoplastic web 30 extending between and connected in abutting relation to adjacent rings 40. The hose 10 may or may not be rendered in a fully extended position prior to annealing.

This particular embodiment of hose includes two or more support rings 40 that form a plurality of ribs of a relatively stiff thermoplastic material, and a thin web or wall 30 of plastic material that extends between the inside diameter, outside diameter, or both, and/or between the rings 40.

An example of an annealing process for treating a hose 10 having individual support rings 40 is depicted in FIG. 7, and comprises:
providing a hose having one or more individual support rings interconnected by a thermoplastic web;
bringing the hose to a fully compressed position such that the individual support rings are brought close together;
heating the hose;
while the hose is hot, extending the hose axially to a partially extended position; and
allowing the hose to cool in the partially extended position.

In an embodiment, the distance between adjacent rings 40 of the hose when the hose is in the partially extended position is between about 20%-95% of the distance between adjacent rings 40 of the hose when the hose is in the fully extended position.

In another embodiment, the distance between adjacent rings 40 of the hose when the hose is in the partially extended position is between about 40%-95% of the distance between adjacent rings 40 of the hose when the hose is in the fully extended position.

In yet another embodiment, the distance between adjacent rings 40 of the hose when the hose is in the partially extended position is between about 60%-95% of the distance between adjacent rings 40 of the hose when the hose is in the fully extended position.

In a further embodiment, the distance between adjacent rings 40 of the hose when the hose is in the partially extended position is between about 60%-80% of the distance between adjacent rings 40 of the hose when the hose is in the fully extended position.

In a still further embodiment, the distance between adjacent rings 40 of the hose when the hose is in the partially extended position is about 80% of the distance between adjacent rings 40 of the hose when the hose is in the fully extended position.

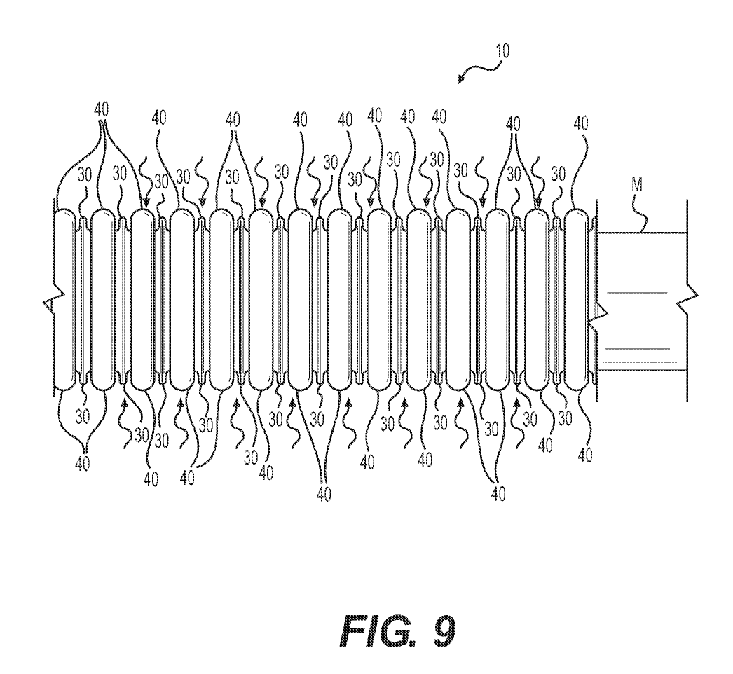

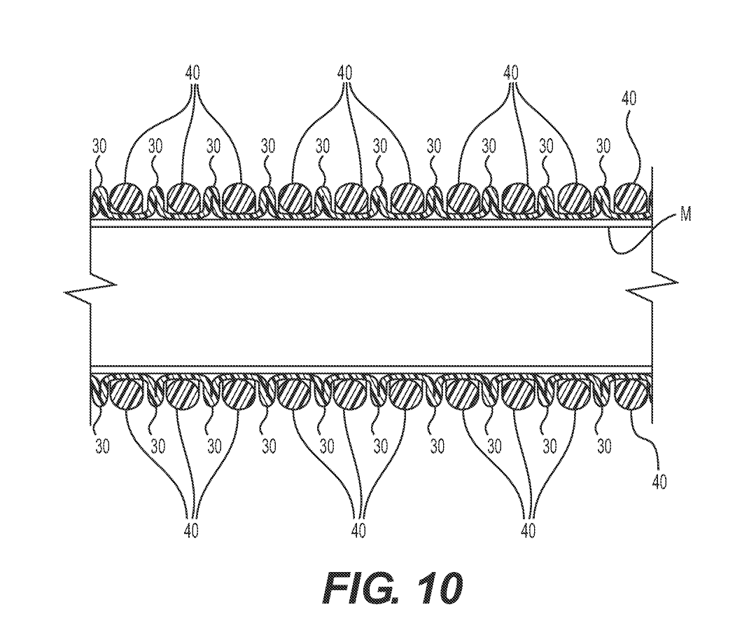

FIG. 8 shows the hose 10 prior to annealing being in a fully-stretched state prior to annealing, although it need not be. FIG. 9-10 show the hose 10 in the fully compressed position during which it is heated to an annealing temperature.

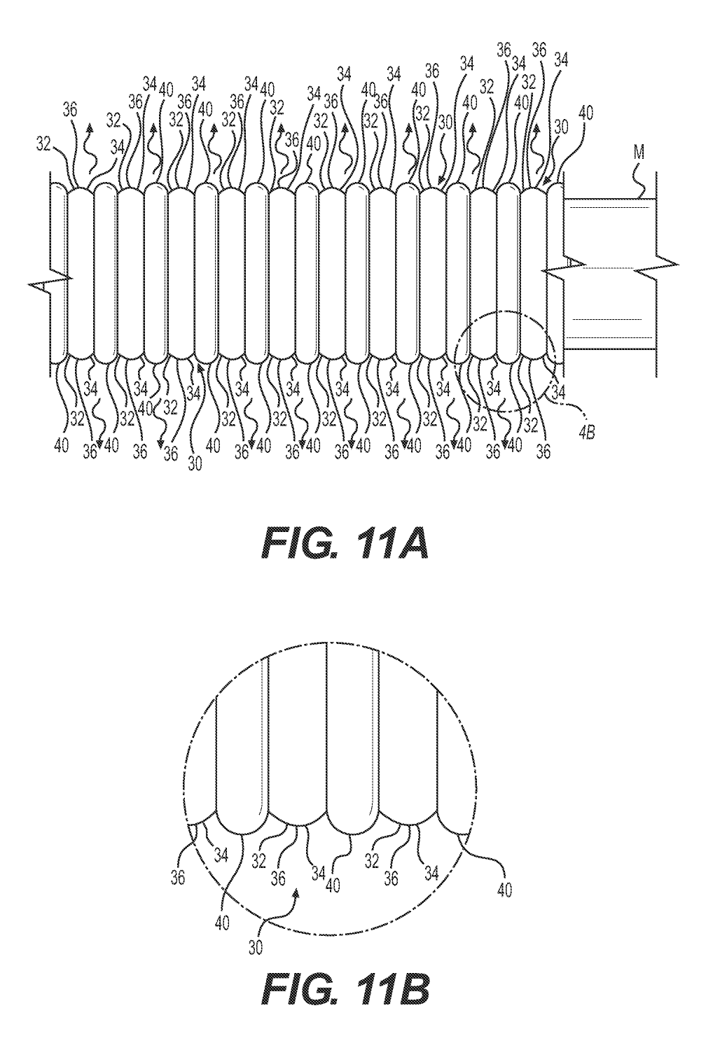

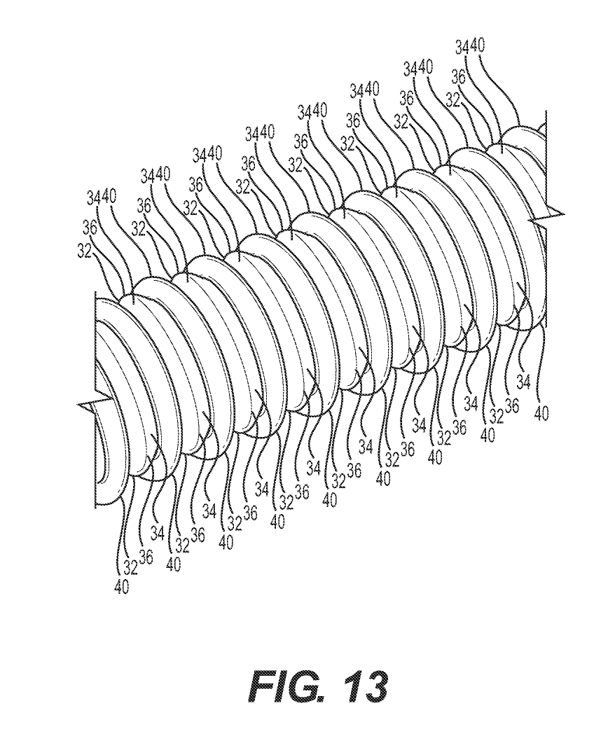

FIGS. 11A and 13 show the hose in the partially-extended position during and after cooling.

FIG. 13 shows the hose 10 in the partially extended or stretched position, which is now its at-rest or equilibrium orientation in the absence of external forces, after being cooled.

It is to be understood that the invention disclosed herein may be employed in and practiced on any hose which employs one or more thermoplastic materials and has one or more stiffening ribs, or alternatively one or more stiffening rings, or both one or more stiffening ribs and stiffening rings.

A still further implementation of the invention comprises a hose 10 having one or more support ribs of any desired number or orientation, including round, oval, helical, randomly shaped or otherwise, and a web of thermoplastic material interconnecting the support ribs, the hose being heat treated by a process comprising:
providing a hose having one or more support ribs interconnected by a thermoplastic web, the hose being in a fully extended orientation;
bringing the hose to a fully compressed position such that the one or more support ribs are brought close together;
heating the hose;
while the hose is hot, extending the hose axially to a partially extended position; and
allowing the hose to cool in the partially extended position.

In an embodiment, the distance between adjacent ribs of the hose when the hose is in the partially extended position is between about 20%-95% of the distance between adjacent ribs of the hose when the hose is in the fully extended position.

In another embodiment, the distance between adjacent ribs of the hose when the hose is in the partially extended position is between about 40%-95% of the distance between adjacent ribs of the hose when the hose is in the fully extended position.

In yet another embodiment, the distance between adjacent ribs 40 of the hose when the hose is in the partially extended position is between about 60%-95% of the distance between adjacent ribs of the hose when the hose is in the fully extended position.

In a further embodiment, the distance between adjacent ribs of the hose when the hose is in the partially extended position is between about 60%-80% of the distance between adjacent ribs of the hose when the hose is in the fully extended position.

In a still further embodiment, the distance between adjacent ribs of the hose when the hose is in the partially extended position is about 80% of the distance between adjacent ribs of the hose when the hose is in the fully extended position.

The hose 10 treated by any of the above described processes can be stretched, bent, twisted and axially compressed without imposing substantial dislocating forces on the associated equipment. Stretching hose 10 causes the windings of helical rib 20, the individual rings 40, or other support rib structure to separate, which thereby causes the outwardly extending portions 32 and 34 (as best seen in FIGS. 4A, 4B, 5A, 5B, 6, 11A, 11B, 12A, 12B and 13) of the web 30 to flatten out or “unfold” about crease line 36. If, during stretching or extension, the hose 10 is caused to bend or deflect, the crush resistant character of the hose 10 will permit the bending or deflection to take place without significantly diminishing the inner diameter of the hose 10.

Axially compressing the hose 10 treated by any of the processes of this invention from the at rest orientation shown in FIGS. 4A-6 and 11A-13 likewise does not result in the diameter of hose 10 being appreciably reduced because of the inherent flexibility of the hose, yet also does not cause the imposition of substantial dislocating forces on the associated equipment.

The annealing process modifies the orientation of the molecules of thermoplastic that forms the coils of the helical rib 20, the individual support rings 40, or other support rib(s), and the thin wall or web 30 that extends therebetween.

In an embodiment, when the hose 10 is formed, the coils of the rib 20, rings 40 or other support rib(s) are relatively widely spaced (for example, the hose 10 prior to treatment may be in its fully extended position or orientation as shown in FIGS. 1 and 8), and the thin web 30 of plastic mate that extends between adjacent windings of the rib 20, rings 40 or other support rib(s) takes a substantially cylindrical shape that may not project radially outwardly at locations between the windings of the rib 20, rings 40 or other support rib(s). However, prior to or at the commencement of the annealing process, the windings of the helical rib 20, rings 40 or other support rib(s) are moved close to each other as shown in MS. 2, 3, 9 and 10, which causes the web 30 situated between adjacent winds of helical rib 20, rings 40 or other support rib(s) to bulge radially outwardly, creating the radially outwardly extending portions denoted by respective left and right web portions 32 and 34, and by crease 36. As the rib 20, rings 40 or other support rib(s) assume the side-by-side positions snugly sandwiching the radially outwardly bulging web 30 therebetween, a reverse-direction crease or fold 36 is caused to form and set at a central location extending circumferentially about the web 30.

The heating and controlled cooling of the annealing process includes heating the compressed hose 10, extending the hose 10 to a partially extended position where the distance between adjacent winds of rib 20 or rings 40 of the hose when the hose is in the partially extended position is between about 20%-95% of the distance between adjacent winds of rib 20 or rings 40 of the hose when the hose is in the fully extended position, and then cooling the hose 10. As this process is carried out, the molecules of the material of the rib 20, rings 40 or other support rib(s), and the web 30, relax and take on a new orientation, with a memory of the partially extended hose 10 being such that the hose 10 stays in the partially extended to which the completed hose 10 will normally return when released from the imposition of external forces. And, because stress is substantially absent from the hose 10 when the hose 10 is in the partially extended position, the hose 10 begins resisting extension or compression only when, and to the extent that, the hose 10 is stretched causing it to lengthen or compressed causing it to shorten. In addition, far greater economy of material is achieved over prior annealed thermoplastic hoses by having the hose be somewhat elongated in its at-rest position, because prior annealed hoses are made to be in their fully compressed orientation when at rest, resulting in far heavier and costlier hoses.

The annealing process to which the hose 10 is subjected allows the hose to exhibit a greater degree of flexibility and an ease of being stretched, compressed and twisted than is exhibited by conventional, non-annealed, hose products, and enables the hose 10 to, in effect, provide a “strain relief” between medical delivery equipment (not shown) that typically is connected to one end of a length of the hose 10, and a patient's facial or nasal mask (not shown) that typically is connected to an opposite end of the same length of hose 10.

Yet another benefit of the annealed and stress-relieved hose 10 (which results from stresses that were introduced during the manufacture of the hose 10 being relieved during annealing) is that the stress-relieved hose 10 does not take a set (i.e., does not take on a configurational memory to which the hose 10 seeks to return) when deflected or bent in any one direction or orientation for a lengthy period of time.

The degree to which the hose 10 of this invention can be stretched or axially compressed depends upon the distance between the winds of helical rib 20, rings 40 or other support rib(s), as well as the partially extended orientation of the hose 10 after annealing. Thus, the distance which the treated hose 10 may be stretched or compressed depends to a large degree on the length of the web sections 32 and 34 on either side of centrally located reverse-direction crease or fold 36. Crease or fold 36 acts as a living hinge between web sections 32 and 34. The longer the length of the web sections 32 and 34, the more the hose 10 may be stretched or compressed, and the further and easier it may be bent angularly, or twisted.

Although the invention has been described in its preferred form with a certain degree of particularity, it is understood that the present disclosure of the preferred form and alternate embodiments has been made only by way of example, and that numerous changes in the details of construction and the manner of manufacture may be resorted to without departing from the spirit and scope of the invention. It is intended to protect whatever features of patentable novelty exist in the invention disclosed. The claims that follow are intended to protect whatever features of patentability that exist in the inventive features disclosed in the text, the drawings and the claims hereof.

What is claimed is:

1. A flexible, stretchable, axially compressible fluid-carrying hose, the hose including one or more relatively rigid thermoplastic support ribs disposed about a periphery of the hose, the hose also including a thermoplastic web extending between and connected in abutting relation to adjacent winds of the one or more support ribs, the annealing process comprising:
bringing the hose to a fully compressed position such that the winds of the rib are brought close together;
heating the hose;
while the hose is hot, extending the hose axially to a partially extended position;
allowing the hose to cool in the partially extended position;
wherein the distance between adjacent winds of the rib when the hose is in the partially extended position is between about 20%-95% of the distance between adjacent winds of the rib when the hose is in a fully extended position.

2. The hose of claim 1, wherein the distance between adjacent winds of the rib when the hose is in the partially extended position is between about 40%-95% of the distance between adjacent winds of the rib when the hose is in the fully extended position.

3. The hose of claim 1, wherein the distance between adjacent winds of the rib when the hose is in the partially extended position is between about 60%-95% of the distance between adjacent winds of the rib when the hose is in the fully extended position.

4. The hose of claim 1, wherein the distance between adjacent winds of the helical rib(s) 20 when the hose is in the partially extended position is between about 60%-80% of the distance between adjacent winds of the helical rib(s) 20 when the hose is in the fully extended position.

5. The hose of claim 1, wherein the distance between adjacent winds of the rib when the hose is in the partially extended position is about 80% of the distance between adjacent winds of the rib when the hose is in the fully extended position.

6. A flexible, stretchable, axially compressible fluid-carrying hose, the hose including one or more relatively rigid thermoplastic support rings disposed about a periphery of the hose, the hose also including a thermoplastic web extending between and connected in abutting relation to adjacent rings, the hose having a fully extended position prior to annealing, the annealing process comprising:
providing a hose having one or more individual support rings interconnected by a thermoplastic web;
bringing the hose to a fully compressed position such that the individual support rings are brought close together;
heating the hose;
while the hose is hot, extending the hose axially to a partially extended position;
allowing the hose to cool in the partially extended position;
wherein the distance between adjacent rings when the hose is in the partially extended position is between about 20%-95% of the distance between adjacent rings when the hose is in a fully extended position.

7. The hose of claim 6, wherein the distance between adjacent rings when the hose is in the partially extended position is between about 40%-95% of the distance between adjacent rings when the hose is in the fully extended position.

8. The hose of claim 6, wherein the distance between adjacent rings when the hose is in the partially extended position is between about 60%-95% of the distance between adjacent rings when the hose is in the fully extended position.

9. The hose of claim 6, wherein the distance between adjacent rings when the hose is in the partially extended position is between about 60%-80% of the distance between adjacent rings when the hose is in the fully extended position.

10. The hose of claim 6, wherein the distance between adjacent rings 40 of the hose when the hose is in the partially extended position is about 80% of the distance between adjacent rings 40 of the hose when the hose is in the fully extended position.

11. A flexible, stretchable, axially compressible fluid-carrying hose, the hose including one or more relatively rigid thermoplastic support ribs disposed about a periphery of the hose, the hose also including a thermoplastic web extending between and connected in abutting relation to adjacent ribs, the hose being in a fully extended position prior to annealing, the annealing process comprising:
providing a hose having one or more support ribs interconnected by a thermoplastic web, the hose being in a fully extended orientation;
bringing the hose to a fully compressed position such that the one or more support ribs are brought close together;
heating the hose;
while the hose is hot, extending the hose axially to a partially extended position between 20% and 95% of its fully extended position;
allowing the hose to cool in the partially extended position;
wherein the distance between adjacent ribs when the hose is in the partially extended position is between about 20%-95% of the distance between adjacent ribs when the hose is in a fully extended position.

12. The hose of claim 11, wherein the distance between adjacent ribs of the hose when the hose is in the partially extended position is between about 40%-95% of the distance between adjacent ribs of the hose when the hose is in the fully extended position.

13. The hose of claim 11, wherein the distance between adjacent ribs 40 of the hose when the hose is in the partially extended position is between about 60%-95% of the distance between adjacent ribs of the hose when the hose is in the fully extended position.

14. The hose of claim 11, wherein the distance between adjacent ribs of the hose when the hose is in the partially extended position is between about 60%-80% of the distance between adjacent ribs of the hose when the hose is in the fully extended position.

15. The hose of claim 11, wherein the distance between adjacent ribs of the hose when the hose is in the partially extended position is about 80% of the distance between adjacent ribs of the hose when the hose is in the fully extended position.
